(19) United States
(12) Patent Application Publication
(10) Pub. No.: US 2007/0166306 A1
(43) Pub. Date: Jul. 19, 2007

(54) Anti-CD19 antibody composition and method

(76) Inventors: Georg H. M. Fey, Neunkirchen a. Br. (DE); Karin Barbin, Furth (DE); Julia Stieglmaier, Erlangen (DE); Matthias Peipp, Hamburg (DE)

Correspondence Address: PERKINS COIE LLP, P.O. BOX 2168, MENLO PARK, CA, 94026, US

(21) Appl. No.: 11/334,003

(22) Filed: Jan. 17, 2006

(51) Int. Cl. A61K 39/395; A61K 31/715

(52) U.S. Cl. 424/141.1; 424/144.1; 514/54

(57) ABSTRACT

A method of enhancing the antibody-dependent cellular cytotoxicity (ADCC) of a human or humanized CD19 antibody is disclosed. The antibody is produced in the presence of a beta.(1,4)-N-acetylglucosaminyltransferase III (GnTIII) enzyme, under conditions effective to produce in the antibody, an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides. Also disclosed is an anti-CD19 antibody, a composition produced by the method, a treatment method for cancers associated with malignant B-lineage cells, such as chronic lymphocytic leukemia, Non-Hodgkin lymphoma, and acute lymphoblastic leukemia, and a treatment method for autoimmune disease.

### FIELD OF THE INVENTION

The present invention relates to an anti-CD19 antibody with enhanced Fc-receptor activity, and to a method of a method of making and using the antibody.

### BACKGROUND OF THE INVENTION

CD19, a cell surface glycoprotein of the immunoglobulin superfamily is a potentially attractive target for antibody therapy of B-lymphoid malignancies. This antigen is absent from hematopoietic stem cells, and in healthy individuals its presence is exclusively restricted to the B-lineage and possibly some follicular dendritic cells (Scheuermann, R. et al. (1995) Leuk Lymphoma 18, 385-397). Furthermore, CD19 is not shed from the cell surface and rarely lost during neoplastic transformation (Scheuermann, 1995). The protein is expressed on most malignant B-lineage cells, including cells from patients with chronic lymphocytic leukemia (CLL), Non-Hodgkin lymphoma (NHL), and acute lymphoblastic leukemia (ALL) (Uckun, F. M. et al. (1988) Blood 71,13-29). Importantly, CD19 is consistently expressed on B-precursor and mature B-ALLs, whereas CD20 is less frequently expressed, particularly on B-precursor ALLs (Hoelzer, D. et al. (2002) Hematology (Am Soc Hematol Educ Program), 162-192). Therefore, only a portion of these patients can be treated with CD20 antibodies. In contrast, the majority of these patients might benefit from treatment with CD19-specific antibodies, if suitable antibodies were available.

A therapeutic anti-CD19 antibody would ideally (i) be a human or humanized antibody, (ii) trigger enhanced antibody-dependent cellular cytotoxicity (ADCC), and (iii) be produced in a mammalian cell system. The present invention is aimed at providing such an antibody, and its use in treating various B-cell malignancies.

### SUMMARY OF THE INVENTION

The invention includes, in one aspect, a method of enhancing the antibody-dependent cellular cytotoxicity (ADCC) of a human or humanized CD19-specific antibody, by producing the antibody in the presence of a beta.(1,4)-N-acetylglucosaminyltransferase III (GnTIII) enzyme, under conditions effective to produce in the antibody, an Fc fragment characterized by asparagine297 (Asn297)-linked oligosaccharides containing (i) at least 60% N-acetylglucosamine bisecting oligosasccharides, and (ii) at least 10% non-fucosylated N-acetylglucosamine bisecting oligosaccharides. The antibody may be produced in a mammalian cell line transfected with (i) the cDNA for the anti-CD19 antibody and (ii) the cDNA for the GnTIII enzyme.

In another aspect, the invention includes a method for treating a subject having a cancer associated with malignant B-lineage cells, such as chronic lymphocytic leukemia, Non-Hodgkin lymphoma, and acute lymphoblastic leukemia. The method includes treating the patient with a human or humanized anti-CD19 antibody having an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides. The antibody administered may be produced in a mammalian cell line transfected with (i) the cDNA for the anti-CD19 antibody and (ii) the cDNA for the GnTIII enzyme.

A treatment method for treating an autoimmune disease, such as multiple sclerosis, rheumatoid arthritis, and SLE is also disclosed. The method includes treating the patient with a human or humanized anti-CD19 antibody having an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides.

In still another aspect of the invention, there is provided a human or humanized anti-CD19 antibody having an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides. The antibody may be produced in a mammalian cell line transfected with (i) the cDNA for the anti-CD19 antibody and (ii) the cDNA for the GnTIII enzyme. The antibody may be further treated with a fucosidase enzyme effective to remove core fucose groups from said oligosaccharides. Also disclosed is a pharmaceutical composition comprising the antibody in an aqueous pharmaceutical carrier.

These and other objects and features of the invention will become more fully apparent when the following detailed description of the invention is read in conjunction with the accompanying drawings.

### BRIEF DESCRIPTION OF THE DRAWINGS

FIG. 1 shows the scheme of N-linked oligosaccharides attached at Asn297 in the CH2 domain of IgG antibodies;

FIGS. 2A and 2B show the expression vectors for chimeric CD19 antibody (2A) and GnTIII enzyme (2B) in mammalian cells;

FIG. 3A and 3B are SDS-PAGE gel patterns showing expression of CD19 chimeric antibody (3A) and GnTIII enzyme (3B) in co-transfected 293T cells;

FIG. 4 shows SDS-PAGE gel patterns of marker proteins (lane 1), and heavy and light chimeric CD19 antibody chains in CD19 antibody produced by 293T cells in the absence (lane 2) and presence (lane 3) of co-transfected GnTIII enzyme;

FIGS. 5A-5D are flow cytometry analyses that show binding of the chimeric CD19 antibody to 293 cells stably transfected with human CD19 cDNA (A and C) but not to untransfected 293 cells. Cells were incubated with the chimeric CD19 antibody (A and B) or chimeric CD19 antibody co-expressed with GnTIII enzyme (C and D).

FIGS. 6A-6D are positive ion-mode MALDI/TOF-MS spectra of oligosaccharides released from CD19 antibodies formed in the absence (A and B) or presence (C and D) of GnTIII, and after digestion with PNGase F (A and C) or Endo H (B and D);

FIGS. 7A and 7B show possible Asn297-linked oligosaccharide structures present in CD19 antibodies produced in 293T cells in the absence (A) and presence (B) of GnTIII;

FIG. 8 shows lysis of CD19-positive ARH-77 target cells by MNC effector cells in the presence of increasing concentrations of various anti-CD19 antibodies, illustrating the higher ADCC activity of the CD19 antibody produced in accordance with the invention; and

FIGS. 9A and 9B are bar graphs showing ADCC activity of various anti-CD19 antibodies, when measured at one of the effector:target (E:T) ratios indicated.

### DETAILED DESCRIPTION OF THE INVENTION

I. Definitions

The following terms have the meaning defined herein, except when indicated otherwise.

Carbohydrate moieties of the present invention will be described with reference to commonly used nomenclature for the description of oligosaccharides. A review of carbohydrate chemistry which uses this nomenclature is found in Hubbard et al. Ann. Rev. Biochem. 50:555-583 (1981). This nomenclature includes, for instance, Man, which represents mannose; GlcNAc, which represents N-acetylglucosamine; Gal which represents galactose; Fuc for fucose; and Glc, which represents glucose. Sialic acids are described by the shorthand notation NeuNAc, for 5-N-acetylneuraminic acid, and NeuNGc for 5-glycolylneuraminic.

The term “glycosylation” means the attachment of oligosaccharides (carbohydrates containing two or more simple sugars linked together e.g. from two to about twelve simple sugars linked together) to a glycoprotein. The oligosaccharides of the present invention are attached to the asparagine297 (Asn297) residue of a CH2 domain of an IgG1 Fc region as N-linked oligosaccharides.

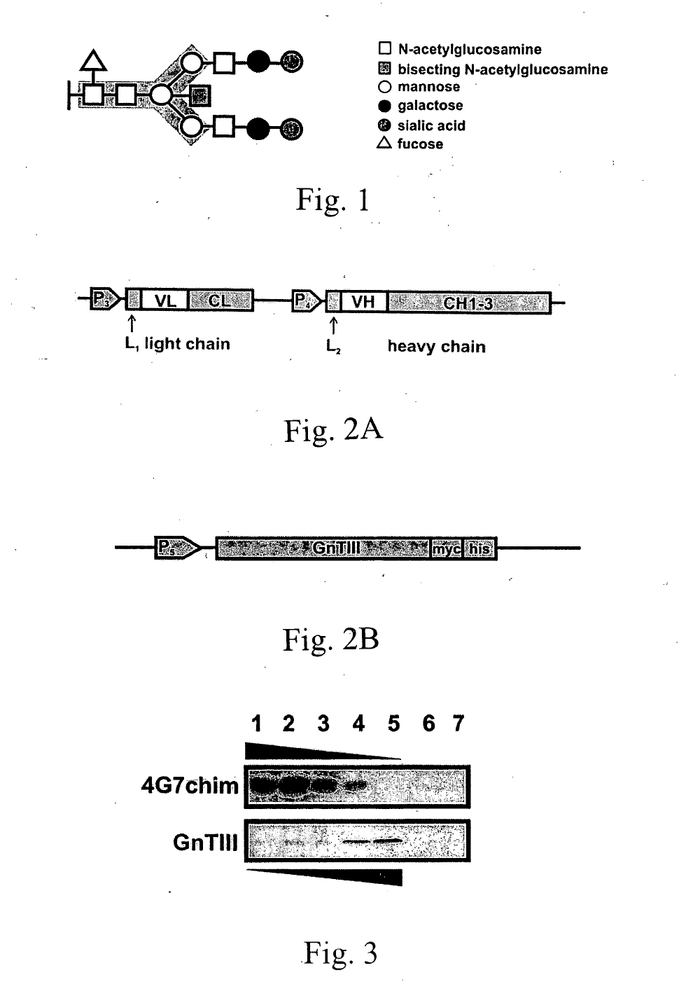

For the purposes herein, a “mature core carbohydrate structure” refers to a processed core carbohydrate structure attached to an Fc region which generally consists of the following carbohydrate structure GlcNAc(Fucose)-GlcNAc-Man-(Man-GlcNAc).sub.2 typical of biantennary oligosaccharides, represented schematically in FIG. 1.

A “bisecting GlcNAc” is a GlcNAc residue attached to the betal,4-linked mannose of the mature core carbohydrate structure. The bisecting GlcNAc can be enzymatically attached to the mature core carbohydrate structure by a beta.(1,4)-N-acetylglucosaminyltransferase III enzyme (GnTIII), typically introduced into a mammalian antibody-expression cell line. A “bisecting oligosaccharide” contains a bisecting GlcNAc.

A “humanized antibody” or “chimeric humanized antibody” shall mean an antibody derived from a non-human antibody, typically a murine antibody, that retains or substantially retains the antigen-binding properties of the parental antibody, but which is less immunogenic in humans.

“GnTIII” enzyme refers to beta.(1,4)-N-acetylglucosaminyltransferase III, as described, for example, in U.S. Pat. No. 6,602,684.

An “anti-CD19 antibody” or “CD19-specific antibody” or “CD19 antibody” refers to an antibody that specifically recognizes the cell-surface glycoprotein of the immunoglobulin superfamily commonly referred to as CD19.

II. Preparation of Anti-CD19 Antibodies with Enhanced ADCC

This section describes the preparation and characterization of anti-CD19 antibodies in accordance with the present invention, that is, anti-CD19 antibodies having an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides. In preferred embodiments, the Asn297-linked oligosaccharides contain (i) at least 70% and as great as 80% or more N-acetylglucosamine bisecting oligosasccharides, and (ii) at least 15% non-fucosylated N-acetylglucosamine bisecting oligosaccharides.

For comparative purposes, and in particular, for demonstrating the enhanced ADCC activity of the novel antibody of the present invention, a CD19-specific antibody was prepared under standard antibody expression conditions in a mammalian cell culture. This antibody is characterized by a substantially lower percentage of N-acetylglucosamine bisecting oligosasccharides, and a substantially lower percentage of non-fucosylated oligosaccharides at the Asn297 position, relative to the antibody of the invention.

The antibodies of the present invention are human or humanized antibodies suitable for human therapy. Humanized antibodies can be prepared based on the sequence of a murine monoclonal antibody prepared according to conventional monoclonal antibody techniques. DNA encoding the heavy and light chain immunoglobulins can be obtained from the murine hybridoma of interest and engineered to contain human immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

More generally, humanized antibodies may be prepared by (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81: 6851-5 (1984); Morrison et al., Adv. Immunol. 44: 65-92 (1988); Verhoeyen et al., Science 239: 1534-1536 (1988); Padlan, Molec. Immun. 28: 489-498 (1991); Padlan, Molec. Immun. 31: 169-217 (1994), and U.S. Pat. Nos. 5,585,089, 5,693,761 and 5,693,762 all of which are hereby incorporated by reference in their entirety.

Human monoclonal antibodies directed against CD19 can be produced using transgenic or transchromosomic mice carrying parts of the human immune system rather than the mouse system. These transgenic and transchromosomic mice include mice referred to herein as the HuMAb Mouse.RTM. and KM Mouse.RTM. respectively, and are collectively referred to herein as “human Ig mice.” The HuMAb Mouse.RTM. (Medarex.RTM., Inc.) contains human immunoglobulin gene miniloci that encode unrearranged human heavy (mu and gamma) and kappa light chain immunoglobulin sequences, together with targeted mutations that inactivate the endogenous .mu. and .kappa. chain loci (see e.g., Lonberg, et al. (1994) Nature 368(6474): 856-859). Accordingly, the mice exhibit reduced expression of mouse IgM or .kappa., and in response to immunization, the introduced human heavy and light chain transgenes undergo class switching and somatic mutation to generate high affinity human IgG.kappa. monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of the HuMab Mouse.RTM., and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-3724; Choi et al. (1993) Nature Genetics 4:117-123; Chen, J. etal. (1993) EMBO J. 12: 821-830; Tuaillon et al. (1994) J. Immunol. 152:2912-2920; Taylor, L. et al. (1994) International Immunology 6:579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al. preferably.

The antibodies are expressed in cells that also express the GnTIII enzyme. Typically, the antibody producing cell has been engineered, e.g., transfected, to contain a GnTIII expression vector constructed for enzyme expression in the host cell. Where the CD19-specific antibody is expressed in a cell line expressing human monoclonal antibodies, this cell line is transfected with suitable GnTIII expression vector. Methods for transfecting and selecting cell lines containing the expression vector are well established. Where the CD19-specific antibody is a chimeric humanized antibody, both the antibody and GnTIII enzyme are co-expressed in a suitable, preferably mammalian cell line that has been transfected, e.g., co-transfected or transfected sequentially with expression vectors for both antibody and enzyme. Methods for constructing antibody and GnTIII expression vectors, for transfecting host cells, and expressing the CD19-specific antibody of the invention in secreted form from host cells are described below and in Examples 1-3.

A. GnTIII Coding Sequence and Expression Vector.

The cDNA encoding GnTIII may be expressed under the control of a constitutive promoter or, alternately, a regulated expression system. Suitable regulated expression systems include, but are not limited to, a tetracycline-regulated expression system, an ecdysone-inducible expression system, a lac-switch expression system, a glucocorticoid-inducible expression system, a temperature-inducible promoter system, and a metallothionein metal-inducible expression system. Methods for constructing an expression vector for GnTIII are detailed in Example 2, with reference to FIG. 2B. Briefly, expression of GnTIII, fused to C-terminal myc- and hexa-histidine tags added for detection, was driven by the CMV promotor.

B. Anti-CD19 Antibody Coding Sequence and Expression Vector

The host cell line expressing GnTIII is also engineered, e.g., by co-transfection, to produce an anti-CD19 antibody, preferably a human or humanized anti-CD19 antibody. One exemplary anti-CD19 antibody coding sequence is detailed in Example 2, with reference to FIG. 2A. Briefly, coding sequences for the variable light (L)-chain of the murine hybridoma antibody 4G7 were fused to sequences coding for the constant region of a human antibody kappa L-chain. Coding sequences for the heavy (H)-chain variable portion of the 4G7 hybridoma antibody were fused to DNA coding for the complete constant region of a human gamma1 H-chain. The anti-CD19 antibody produced in this example is also referred to herein as 4G7chim (for chimeric). Expression of these chimeric antibody chains in mammalian cells was controlled by the human elongation factor 1α (EF-1α) and the human cytomegalovirus immediate-early (CMV) promotors. Secretion of H- and L-chains was enabled by the respective human Ig leader sequences.

C. Transfected Cells

Generally, any type of cultured cell line can be used as a background to engineer the host cell lines of the present invention. In a preferred embodiment, CHO cells, BHK cells, NSO cells, SP2/0 cells, or a hybridoma cell line is used as the background cell line to generate the engineered host cells of the invention. One exemplary cell line employed herein is the human embryonal kidney cell line 293T. Methods for culturing the cells are given in Example 1.

Methods which are well known to those skilled in the art can be used to construct expression vectors containing the coding sequence of the protein of interest and the coding sequence of the glycoprotein-modifying glycosyltransferase and appropriate transcriptional/translational control signals. These methods include in vitro recombinant DNA techniques, synthetic techniques and in vivo genetic recombination. See, for example, the techniques described in Maniatis et al, 1989, Molecular Cloning A Laboratory Manual, Cold Spring Harbor Laboratory, N.Y. and Ausubel et al., 1989, Current Protocols in Molecular Biology, Greene Publishing Associates and Wiley Interscience, N.Y. In the present invention, host cells are transfected with expression vectors for coding for both GnTIII and the anti-CD19 antibody, substantially as described in Example 3.

D. Expression of Anti-CD19 Antibody

Co-transfection of 293T cells with mixtures of expression constructs for anti-CD19 antibody and GnTIII, combined in different proportions, led to the successful simultaneous expression of both proteins, as analyzed by Western blot analysis (FIG. 3). The electrophoretic mobilities of 65 and 53 kDa corresponded to the expected molecular weights for GnTIII and the H-chain of 4G7chim, respectively. Expression levels of both proteins were proportional to the relative amounts of the corresponding cDNAs in the transfected DNA-mixtures.

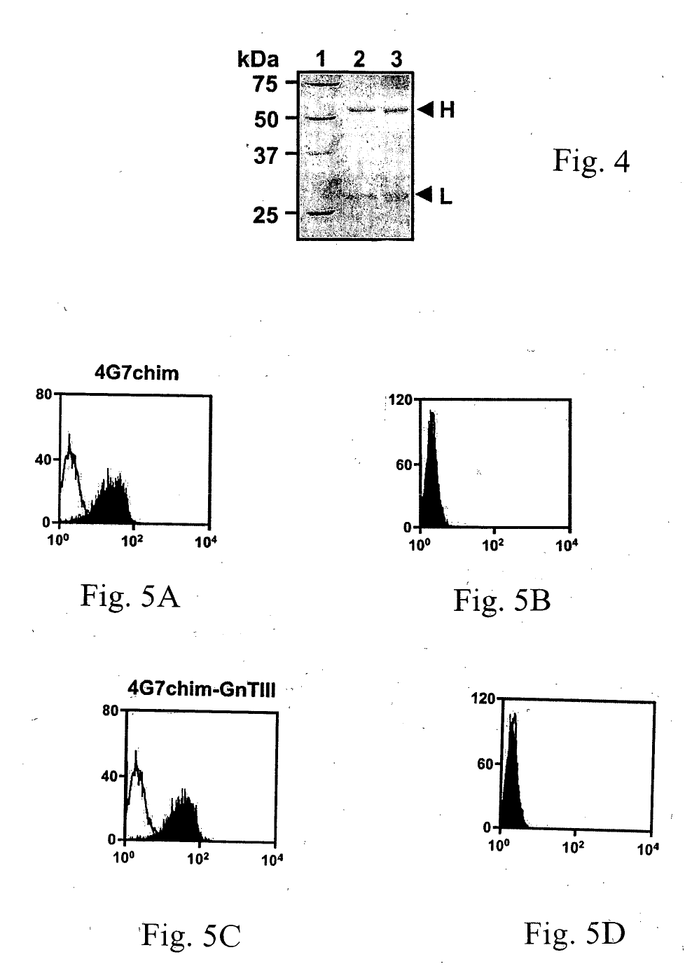

4G7chim expressed in 293T cells, and the modified version from glyco-engineered 293T cells, 4G7chim-GnTIII, were purified from culture supernatants by protein A chromatography. This one-step procedure delivered highly enriched antibody preparations from mammalian cells, with H- and L-chains of the expected size, respectively (FIG. 4). The antibody concentrations were determined by sandwich-ELISA. Yields routinely ranged from 300-400 μg of 4G7chim and 4G7chim-GnTIII per liter of culture supernatant.

The novel CD19-specific antibody of the invention, when carried in a suitable aqueous medium, e.g., a sterile, injectable physiological salt medium, constitutes another aspect of the invention.

E. Binding Properties of anti-CD19 Antibody

The binding properties of the antibodies 4G7chim and 4G7chim-GnTIII were examined by flow cytometric analysis and binding equilibrium studies, employing the methods given in Examples 4A and 4B, respectively. The determination of antibody equilibrium constants was carried out as described in Example 4B. To test specific binding stably transfected 293 cells expressing human CD19 and untransfected 293 cells were used. SEM cells (B-precursor ALL-derived cell line) were used for determination of the equilibrium constants. To exclude a possible influence of the glycosylation status on the binding properties of the chimeric antibodies, their equilibrium constants were determined by a flow cytometry-based method (Table I). As seen in FIG. 5, both antibodies bound specifically to CD19-positive cells, but not to antigen-negative cells (FIG. 5).

TABLE I

| | | | K_{D} ± SEM |
|---|---|---|---|
| antibody | cell line | GnTIII | [×10⁻⁹M] |
| 4G7chim | 293T | − | 6.6 ± 1.1 |
| 4G7chim-GnTIII | 293T | + | 6.6 ± 1.9 |

The K_{D}-values were 6.6±1.1×10⁻⁹ M for 4G7chim and 6.6±1.9×10⁻⁹ M for 4G7chim-GnTIII, respectively. The parental murine antibody 4G7 displayed a K_{D}, value of 9.5±2.8×10⁻⁹ M. Therefore, neither the chimerization nor the expression of the chimeric CD19 antibodies in different systems resulted in significant changes in antibody avidities.

F. Characterization of the Oligosaccharide Composition of the CD19 Antibody

The N-glycan structures of the chimeric CD19 antibodies produced in human 293T cells co-transfected with the GnTIII coding sequence were investigated by enzymatic release of the oligosaccharide followed by MALDI/TOF-MS analyses in the positive ion mode. Under these conditions samples were measured as hydrolyzed, positively charged sodium adducts, and peaks of the resulting MALDI/TOF spectra were assigned to the corresponding oligosaccharide structures. However, some peaks could not be assigned to one defined oligosaccharide structure, as they potentially represented complex, high mannose or hybrid N-glycans of equal masses. To discriminate between these structures, oligosaccharide analyses were performed by digestion with two different enzymes. First, PNGase F was used, which cleaves asparagine-linked complex, hybrid or high mannose oligosaccharides unless alpha.1,3-fucosylated. Second, N-glycans were released with Endo H, which is specific for oligomannose and most hybrid N-glycans, but does not release complex-type glycans. Endo H cleaves between the GlcNAc residues of the chitobiose core, leaving one GlcNAc attached to the asparagine, thereby resulting in a shift of the mass-to-charge ratio (m/z) in MALDI/TOF spectra. Glycosylation in antibody variable regions has been reported to occur (Wright, A. et al. (1993) Springer Semin Immunopathol 15, 259-273). However, the aminoacid sequence of the chimeric CD19 antibody contained no other consensus N-glycosylation site besides Asn297 in the H-chain. Therefore, resulting glycosylation patterns were attributed to carbohydrate structures attached to the antibody Fc portion at Asn297.

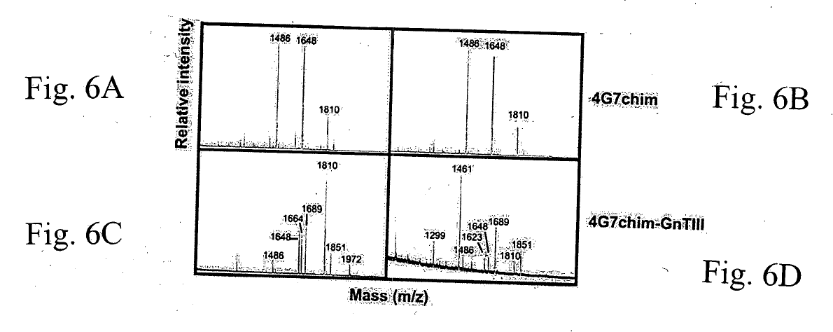

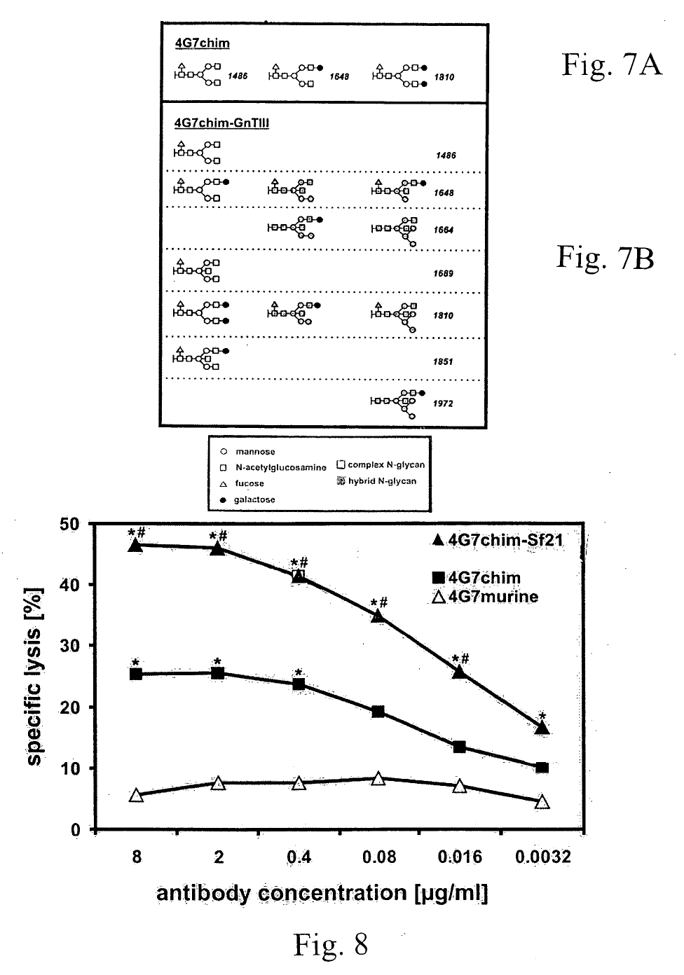

For 4G7chim three dominant peaks were detected (m/z 1486, 1648 and 1810) after both PNGase F and Endo H digestion (FIG. 7A). These were assigned to the corresponding fucosylated biantennary complex oligosaccharide structures (FIG. 6B). Spectra for 4G7chim-GnTIII after PNGase F digestion revealed four main peaks (m/z 1648, 1664, 1689 and 1810) and three minor peaks (m/z 1486, 1851 and 1972). Some of these peaks were ascribed to complex (m/z 1486) or complex bisecting (m/z 1689, 1851) carbohydrate structures, because Endo H digestion did not change the MALDI/TOF spectra. However, cleavage of N-glycans with Endo H resulted in a reduction of the signals detected at m/z values of 1648, 1664, 1810 and 1972. Simultaneously, peaks appeared at m/z values of 1299, 1461 and 1623, indicating the presence of hybrid-type oligosaccharides (FIG. 7B). Thus, co-expression of the chimeric CD19 antibody and GnTIII in 293T cells resulted in the production of antibodies carrying complex and hybrid N-glycans with an increased proportion of bisecting oligosaccharides. Quantitative evaluation of the MALDI/TOF spectra revealed that 4G7chim-GnTIII contained approximately an 8-fold higher proportion of bisecting oligosaccharides compared to 4G7chim (78.7% vs. 9.5%). In addition, co-expression of GnTIII increased the portion of non-fucosylated N-glycans. In particular, bisecting oligosaccharides attached to 4G7chim-GnTIII contained approximately 30-fold more non-fucosylated structures compared to 4G7chim (15.9% vs. 0.5%) (Table II).

TABLE II

| | |
|---|---|
| % bisecting | % non-bisecting |

| antibody | +fuc | −fuc | +fuc | −fuc | sum [%] |
|---|---|---|---|---|---|
| 4G7chim | 9.0 | 0.5 | 84.2 | 8.9 | 102.6 |
| 4G7chim- | 62.8 | 15.9 | 14.3 | 7.0 | 100.0 |
| GnTIII | | | | | |

G. Cytolytic Activity of the Chimeric CD19 Antibody Variants

To investigate the influence of the different oligosaccharide structures on the cytotoxic potential of the chimeric antibodies, comparative ADCC experiments were performed with 4G7chim and 4G7chim-GnTIII. For this purpose the CD19-positive cell line ARH-77 was used as a target in a 3 hr ⁵¹Cr release assay with freshly isolated, unstimulated MNCs as effectors (FIG. 8). Without any added antibody or with the parental murine IgG1 antibody 4G7 no specific lysis was observed. Addition of the chimeric CD19 antibodies triggered specific lysis of target cells in a dose-dependent manner with saturation reached at antibody concentrations between 0.4 and 2 μg/ml (FIG. 8). As seen, 4G7chim-GnTIII triggered stronger ADCC than 4G7chim (p<0.05). Thus, co-expression with GnTIII, under conditions effective to produce in the CD19 antibody, an Fc fragment characterized by Asn297-linked oligosaccharides containing (i) at least 60% N-acetylglucosamine bisecting oligosasccharides, and (ii) at least 10% non-fucosylated bisecting oligosaccharides, significantly enhanced the cytolytic efficacy of the antibody.

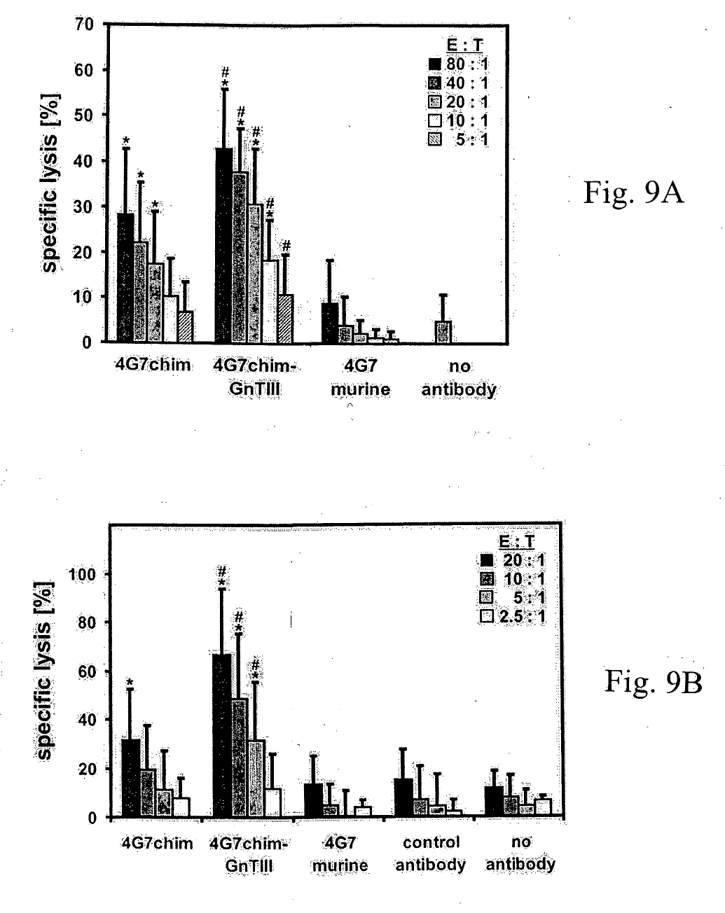

Furthermore, the chimeric CD19 antibodies induced target cell lysis over a broad range of effector-to-target cell (E:T) ratios (FIG. 9A). Whereas 4G7chim-mediated ADCC was significantly enhanced at E:T ratios of 20:1 and higher, 4G7chim-GnTIII triggered increased lysis also at an E:T ratio of 10:1 (p<0.05). By contrast, the murine 4G7 antibody did not mediate lysis at any of these E:T ratios. The 4G7chim-GnTIII antibody displayed stronger cytotoxic potential than 4G7chim at all tested E:T ratios. In conclusion, 4G7chim-GnTIII was more effective in mediating ADCC than 4G7chim, and this difference was attributed to different N-glycan structures, as the avidities of both antibodies were comparable.

The cytotoxic potential of the CD19 antibody of the present invention was tested against cryopreserved primary leukemic blasts from pediatric patients with common ALL (cALL; FIG. 9B). Purified NK cells isolated from healthy unrelated donors were used as effectors. The murine 4G7 hybridoma antibody did not mediate target cell lysis, thus confirming that binding to CD19 alone was not sufficient to induce a cytotoxic effect. Incubation with a chimeric IgG control antibody also did not result in enhanced ADCC. Addition of 4G7chim produced a statistically significant increase in lysis only at an E:T ratio of 20:1 (p<0.05). In contrast, 4G7chim-GnTIII induced statistically significant increased target cell lysis down to E:T ratios of 5:1 (p<0.05). Thus, the CDl9 antibody of the present invention enhanced ADCC both against the leukemia-derived CD19-positive cell line ARH-77, and against primary blasts from pediatric cALL patients.

III. Therapeutic Method

The antibody and antibody composition of the invention are useful in treating a human subject having a cancer associated with malignant B-lineage cells, such as chronic lymphocytic leukemia, Non-Hodgkin lymphoma, and acute lymphoblastic leukemia, as evidenced by enhanced ADCC activity of the antibody against B-lineage cells, described above. In this immunotherapy approach, a patient diagnosed with a cancer associated with malignant B-lineage cells is treated by administration of the anti-CD19 antibody. Preferably the antibody is administered by IV injection in a suitable physiological carrier. The antibody dose is preferably 1 to 10 mg/injection, and the patient is treated at intervals of every 14 days or so. During treatment, the patient is monitored for change in status of the cancer, typically by standard blood cell assays. The treatment may be carried out in combination with other cancer treatments, including drug or radio-isotope therapy, and may be continued until a desired improvement in patient condition is attained.

The CD19 antibody is also useful in treating an autoimmune disease, such as multiple sclerosis, rheumatoid arthritis, and SLE. In this method, a patient diagnosed with an autoimmune disease is treated by administration of the anti-CD19 antibody. Preferably the antibody is administered by IV injection in a suitable physiological carrier. The antibody dose is preferably 1 to 10 mg/injection, and the patient is treated at intervals of every 14 days or so. During treatment, the patient is monitored for improvement in status, e.g., reduced level of pain or discomfort associated with the condition. The treatment may be carried out in combination with other treatments, such as treatment with immunosuppressive drugs, and may be continued until a desired improvement in patient condition is attained, or over an extended period to alleviate symptoms.

The following examples illustrate, but are in no way intended to limit the invention.

### EXAMPLE 1

### Culture of Eukarvotic Cells

Human 293T embryonal kidney cells, 293 cells and 293 cells stably expressing human CD19 were cultured in DMEM-Glutamax-l medium (Invitrogen, Karlsruhe, Germany) supplemented with 10% fetal calf serum (FCS), 1% penicillin and streptomycin (Invitrogen). ARH-77 (EBV-transformed B-lymphoblastoid cell line established from a patient with plasma cell leukemia; from the American Type Culture Collection, ATCC) and SEM cells, derived from a patient with B-precursor ALL (Greil et al. (1994) Br J Haematol 86, 275-283), were cultured in RPMI 1640-Glutamax-l medium (Invitrogen), containing 10% FCS, 1% penicillin and streptomycin (Invitrogen).

### EXAMPLE 2

### CD19-specific Antibody and GnTIII Expression Vectors

Expression vector for the chimeric CD19-specific IgG1 antibody. Coding regions for the CD19 chimeric antibody were derived from a vector containing (i) a fusion construct coding for a human immunoglobulin heavy (H) chain secretion leader; the variable region of the murine CD19 antibody 4G7 (Meeker et al. (1984) Hybridoma 3, 305-320), and the complete constant region of human gamma1 heavy chain, framed by BamH I restrictions sites. The light (L) chain sequence, consisting of a human immunoglobulin L-chain secretion leader, the variable region of the murine 4G7 antibody and a human kappa L-chain constant domain, was framed by Bgl II restriction sites. The H- and L-chain coding sequences were excised from this vector and inserted into the mammalian expression vector pBud/CHO (unpublished data) derived from the vector pBud CE4.1 (Invitrogen). The coding sequence for the antibody L-chain including the leader was excised from the vector by digestion with Bgl II and was then inserted into the BamH I restriction site. Subsequently, the H-chain of the chimeric antibody with the corresponding human H-chain leader was excised by BamH I digestion from the vector and inserted into the Bgl II restriction site, resulting in the mammalian expression vector pBud/4G7chim.

Expression vector for GnTIII. The cDNA sequence coding for rat GnTIII (GnTIII) was obtained from rat liver polyA mRNA (BD Biosciences Clontech, Palo Alto, Calif., USA) by reverse transcription using standard procedures. The GnTIII coding sequence was amplified with the 5′primer GnTIII for (5′-ACG TGC TAG CCA CCA TGA GAC-3′), containing an Nhe I restriction site, and the 3′primer GnTIII back (5′-ACG TTT CTA GAT GGC CCT CCG-3′), containing an Xba I restriction site. The GnTIII fragment was digested with Nhe I and Xba I and inserted into the Nhe I/Xba I digested vector pSecTag2HygroC-GFP+/anti-CD19 HD37 scFv (Peipp et al. (2004) J Immunol Methods 285, 265-280), thereby generating the expression vector pSecTag2HygroC/GnTIII. This vector enabled the intracellular expression of GnTIII fused to a C-terminal myc-tag and hexa-histidin tag, added for detection of the recombinant protein.

### EXAMPLE 3

### Expression and Purification of Anti-CD19 Antibody

For mammalian expression, 293T cells were transiently transfected using the calcium phosphate method including the addition of 50 μM chloroquine to the transfection mix (Sambrook. J., and Russel. D. W. (2001) Molecular Cloning: A Laboratory Manual. 3 Ed., Cold Spring Harbor Laboratory Press, Cold Spring Harbor, NY.). Transfection was performed with a total of 20 μg plasmid DNA per 100 mm culture dish, containing either the cDNA coding for 4G7chim alone or in combination with cDNA for GnTIII. After 9 h, transfection medium was replaced by fresh medium. After 48 h medium was exchanged, and culture supernatants were collected for five consecutive days. Purification of the secreted antibodies from culture supernatants was performed by affinity chromatography with protein A agarose (Sigma-Aldrich, Taufkirchen, Germany) according to manufacturer's instructions. For oligosaccharide analyses, the chimeric antibodies produced in 293T cells were purified via protein A chromatography on a 1 ml HiTrap protein A column (Amersham, Freiburg, Germany), using a pH gradient elution with a 100 mM phosphate-citrate buffer (pH7 to pH3), to eliminate bovine IgG.

A. Determination of Antibody Concentration by Sandwich-ELISA

Antibody concentrations were determined following published procedures Briefly, dilutions of a standard human IgG antibody (Sigma) with defined concentrations and dilutions of the purified antibody were incubated in EIA/RIA microplates (Corning, Wiesbaden, Germany), previously coated with a rabbit anti-human kappa chain antibody (DakoCytomation, Hamburg, Germany). Bound antibody was detected with a horseradish peroxidase-conjugated goat anti-human Fc antibody (Sigma), following development using the ABTS reagent (Roche Diagnostics, Mannheim, Germany). A standard curve was generated and relative to this curve concentrations of purified protein samples were determined.

B. Cell Lysates, SDS-PAGE and Western Blot Analysis

Two days after transfection with expression constructs for 4G7chim alone or in combination with GnTIII, cells were washed once with PBS and cell lysates were prepared by resuspension of 5 million cells in 100 μl lysis buffer (50 mM Tris HCl pH8, 150 mM NaCl, 0.02% NaN₃, 1% TritonX, 0.1% SDS) containing Complete™ Mini proteinase inhibitor (Roche Diagnostics). After incubation on ice for 30 minutes and vortexing, cell debris were removed by centrifugation at 4° C. and protein concentrations were determined using Bradford Reagent (Sigma). SDS-PAGE under reducing conditions was performed according to standard procedures (Sambrook, supra). 4G7chim was detected with a horseradish peroxidase-coupled secondary antibody against human IgG heavy chains (Sigma), GnTIII with a penta-histidin antibody (Qiagen) and a horseradish peroxidase-conjugated secondary antibody according to manufacturer's protocols. Western blots were developed using enhanced chemiluminescence reagents (Amersham).

### EXAMPLE 4

### Characterization of Antibody Binding Properties

A. Flow Cytometric Analysis

For immunofluorescence analysis of the chimeric antibody, cells were incubated with the purified recombinant protein (1 μg/ml) or human IgG (Sigma) as an isotype control for 30 min on ice. After washing with PBS containing 0.1% bovine serum albumin and 7 mM sodium azide, a FITC-conjugated anti-human IgG (Sigma) was used as secondary antibody. Flow cytometry was performed on a FACSCalibur instrument with CellQuest software (Becton Dickinson, Heidelberg, Germany). For each sample 1×10⁴ events were collected and analyses of whole cells were performed using appropriate scatter gates to exclude cellular debris and aggregates.

B. Determination of Antibody Equilibrium Constants (K_{D})

K_{D} values were determined by flow cytometry using published procedures (Benedict, C. A. et al. (1997) J Immunol Methods 201, 223-231). Experiments were repeated 6 times and mean values are reported. Values and graphical analyses were generated using GraphPad Prism Software (GraphPad Software Inc., San Diego, Calif., USA).

### EXAMPLE 5

### Glycosylation Analyses

Oligosaccharide analyses were performed by matrix-assisted laser desorption/ionization time-of-flight mass spectrometry (MALDI/TOF-MS) in the positive ion mode using a 2,5-dihydrobenzoic acid (DHB) matrix following published protocols (Papac, D. I. et al. (1998) Glycobiology 8, 445-454). Briefly, purified antibody samples were dialyzed against 2 mM Tris pH7.0 and concentrations were adjusted to 2 μg/pl. N-linked oligosaccharides were released by digestion with peptide N-glycosidase F (PNGase F). In addition, digestion with endoglycosidase H (Endo H) was performed to discriminate between complex and hybrid N-glycan structures. Samples were analyzed by MALDI/TOF-MS using an Autoflex spectrometer (Bruker Daltonics, Billerica. Mass., USA).

### EXAMPLE 6

### Cytotoxicity Studies

A. Isolation of Mononuclear Cells (MNCs)

20 ml of peripheral blood was obtained from healthy volunteers and MNCs were isolated as described (Elsasser, D. et al. (1996) Blood 87, 3803-3812).

Purity of MNCs was assessed by cytospin preparations and exceeded 95%. Viability of cells was >95% as tested by trypan blue exclusion.

B. Positive Selection of CD56-positive Cells

Peripheral mononuclear cells were enriched by immunomagnetic separation with CD56 microbeads (Miltenyi Biotec) as described (Lang, P. et al. (2002) BoneMarrow Transplant 29, 497-502).

C. Cytotoxicity Experiments

ADCC assays with MNCs from healthy donors as effector cells were performed by a 3 h ⁵¹Cr release assay as described (Elsasser, supra), using ARH-77 cells as targets. Cytotoxicity experiments with purified NK cells as effectors and cryopreserved primary common ALL (cALL) blasts as target cells were performed in a 2 h BATDA (bis (acetoxymethyl)2,2′:6′,2″-terpyridine-6,6″-dicarboxylate)europium release assay as previously described. (Lang P. et al. (2004) Blood 103, 3982-3985). All ADCC assays were performed in triplicates.

D. Statistical Analyses

Group data are reported as mean values±standard error of the mean (SEM). Differences between groups were analyzed by paired (or, when appropriate, unpaired) Student's t-test.

Although the invention has been described with respect to particular embodiments and applications, it will be appreciated that various changes and modifications may be made without departing from the invention as claimed.

It is claimed:

1. A method of enhancing the antibody-dependent cellular cytotoxicity (ADCC) of a human or humanized CD19 antibody, comprising
producing the antibody in the presence of a beta.(1,4)-N-acetylglucosaminyltransferase III (GnTIII) enzyme, under conditions effective to produce in the antibody, an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides.

2. The method of claim 1, wherein the antibody is produced in a mammalian cell line transfected with (i) the cDNA for the anti-CD19 antibody and (ii) the cDNA for the GnTIII enzyme.

3. A method for treating a subject having a cancer associated with malignant B-lineage cells, such as chronic lymphocytic leukemia, Non-Hodgkin lymphoma, and acute lymphoblastic leukemia, comprising
treating the patient with a human or humanized anti-CD19 antibody having an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides.

4. The method of claim 3, wherein the antibody administered is produced in a mammalian cell line transfected with (i) the cDNA for the anti-CD19 antibody and (ii) the cDNA for the GnTIII enzyme.

5. A method for treating an autoimmune disease, such as multiple sclerosis, rheumatoid arthritis, and SLE, comprising
treating the patient with a human or humanized anti-CD19 antibody having an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides.

6. A human or humanized anti-CD19 antibody having an Fc fragment characterized by Asn297-linked oligosaccharides containing (i) at least 60% N-acetylglucosamine bisecting oligosasccharides, and (ii) at least 10% non-fucosylated N-acetylglucosamine bisecting oligosaccharides.

7. The antibody of claim 6, which is produced in a mammalian cell line transfected with (i) the cDNA for the anti-CD19 antibody and (ii) the cDNA for the GnTIII enzyme.

8. The antibody of claim 7, which is treated with a fucosidase enzyme effective to remove core fucose groups from said oligosaccharides.

9. A pharmaceutical composition comprising the antibody of claim 6 in an aqueous pharmaceutical carrier.
